(19) United States
(12) Patent Application Publication
(10) Pub. No.: US 2013/0126388 A1
(43) Pub. Date: May 23, 2013

(54) MEDICAL PRODUCT PACKAGE ASSEMBLY

(71) Applicant: Anne Hannahan, St. Paul, MN (US)

(72) Inventor: Anne Hannahan, St. Paul, MN (US)

(21) Appl. No.: 13/683,809

(22) Filed: Nov. 21, 2012

Related U.S. Application Data
(60) Provisional application No. 61/563,364, filed on Nov. 23, 2011.

(51) Int. Cl. A61F 13/551

(52) U.S. Cl. CPC A61F 13/55145
USPC 206/570

(57) ABSTRACT

A medical product package assembly includes a package having an interior flexible sheet disposed between two exterior flexible sheets, the interior and exterior flexible sheets coupled together to form a first compartment and a second compartment wherein the first compartment and the second compartment are adjacently disposed and are formed on opposite sides of the interior flexible sheet, and a releasable seal path extends across a length of the first and second compartments.

### CROSS-REFERENCE TO RELATED APPLICATIONS

This application claims priority under 35 U.S.C. §119(e) to U.S. Provisional Patent Application Ser. No. 61/563,364 filed on Nov. 23, 2011, and incorporated herein by reference.

### BACKGROUND

Urinary incontinence, fecal incontinence, and menses affect numerous men and women. According to the International Continence Society (ICS), urinary incontinence affects 200 million people worldwide. ICS states that 25 million adult Americans experience transient or chronic urinary incontinence and that 75-80% of those sufferers are women. Consumer research reveals that one in four women over the age of 18 experience episodes of leaking urine involuntarily. Some studies show that a third of men and women ages 30-70 have experienced loss of bladder control at some point in their adult lives. Examples of adults effected are women experiencing menses, postpartum women with bloody discharge, postmenopausal women experiencing urinary incontinence, people experiencing stress incontinence, urge incontinence and fecal incontinence, people with overactive bladder, men with an enlarged prostate or Benign Prostatic Hyperplasia (BPH), and men who have had prostate surgery and are experiencing incontinence.

The incidence of these medical conditions and incontinence can occur when people are traveling or otherwise away from their homes. Towelettes and disposable pads and undergarments are often used to address issues of odor protection and cleanliness in regard to urine, feces, and menses resulting from medical conditions. Existing packaging of towelettes and disposable pads and undergarments include multi-packs of each respective type of item sold individually (i.e., towelettes are sold in a multi-pack of towelettes). Existing packaging is bulky and made for multiple uses.

### SUMMARY

A medical product package assembly includes a package having an interior flexible sheet disposed between two exterior flexible sheets, the interior and exterior flexible sheets coupled together to form a first compartment and a second compartment wherein the first compartment and the second compartment are adjacently disposed and are formed on opposite sides of the interior flexible sheet, and a releasable seal path extending across a length of the first and second compartments.

### BRIEF DESCRIPTION OF THE DRAWINGS

The accompanying drawings are included to provide a further understanding of embodiments and are incorporated in and constitute a part of this specification. The drawings illustrate embodiments and together with the description serve to explain principles of embodiments. Other embodiments and many of the intended advantages of embodiments will be readily appreciated as they become better understood by reference to the following detailed description. The elements of the drawings are not necessarily to scale relative to each other. Like reference numerals designate corresponding similar parts.

FIG. 1 illustrates front view of a medical product package assembly according to one embodiment.

FIG. 2 illustrates side cross-sectional view of the medical product package assembly illustrated in FIG. 1 according to one embodiment.

FIG. 3 illustrates front view of a medical product package assembly according to one embodiment.

FIG. 4 illustrates side cross-sectional view of the medical product package assembly illustrated in FIG. 3 according to one embodiment.

FIG. 5 illustrates side cross-sectional view of the medical product package assembly according to one embodiment.

FIG. 6 illustrates a front view of the medical product package assembly according to one embodiment.

FIG. 7 illustrates a back view of the medical product package assembly according to one embodiment.

FIG. 8 illustrates a side cross-sectional view of the medical product package assembly according to one embodiment.

FIG. 9 illustrates a front view of a medical product package assembly according to one embodiment.

FIG. 10 illustrates a front view of a medical product package assembly according to one embodiment.

FIG. 11 is a block diagram of a method of using a medical product package assembly according to one embodiment.

### DETAILED DESCRIPTION

In the following Detailed Description, reference is made to the accompanying drawings, which form a part hereof, and in which is shown by way of illustration specific embodiments in which the invention may be practiced. In this regard, directional terminology, such as “top,” “bottom,” “front,” “back,” “leading,” “trailing,” etc., is used with reference to the orientation of the Figure(s) being described. Because components of embodiments can be positioned in a number of different orientations, the directional terminology is used for purposes of illustration and is in no way limiting. It is to be understood that other embodiments may be utilized and structural or logical changes may be made without departing from the scope of the present invention. The following detailed description, therefore, is not to be taken in a limiting sense, and the scope of the present invention is defined by the appended claims.

It is to be understood that the features of the various exemplary embodiments described herein may be combined with each other, unless specifically noted otherwise.

Embodiments provide a package or container having a plurality of compartments that can each hold its own product and releasably sealed for keeping the products separate and enveloped within their respective compartments until use of the products are desired. Embodiments of the package can be resealable after use of the products and can be used to contain and dispose of soiled or otherwise used products. Embodiments of the package are suitably sized to accommodate the products to be housed within. The products assembled in the package can include items such as a disposable absorbent pad, disposable undergarments, and towelettes, for example. Embodiments provide a discreet and mobile package of medical products.

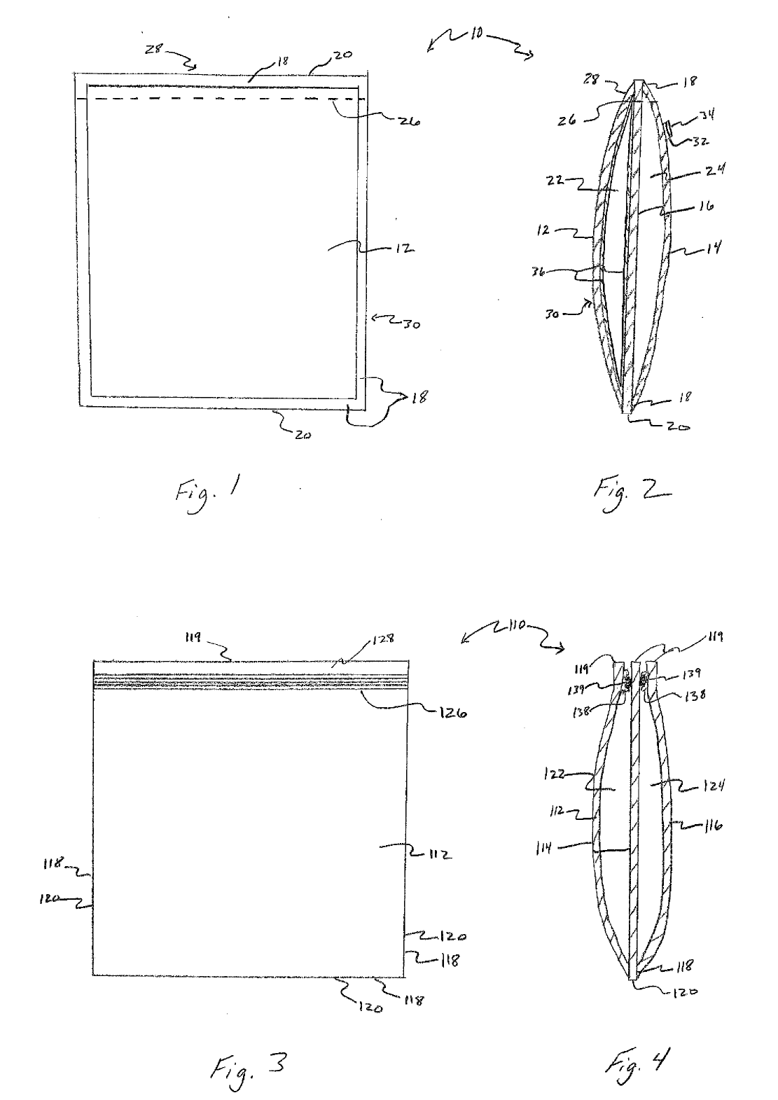

FIGS. 1 and 2 illustrate a medical product package 10 according to one embodiment. The package includes outer flexible sheets 12, 14 arranged on opposite sides of inner flexible sheet 16. The flexible sheets 12, 14, 16 are arranged adjacent one another and a seal 18 is formed along the periphery edge 20 of each of the three overlying flexible sheets 12, 14, 16 to produce the package 10 having a first compartment 22 and a second compartment 24. The seal 18 can be formed by application of an adhesive, by heat and pressure, by laser welding, or by a suitable attaching process or combination of processes known in the art. With reference to FIG. 2, the inner flexible sheet 16 separates the first compartment 22 from the second compartment 24 of package 10.

A releasable seal path 26 is located parallel to, and inset from, the seal 18 along at least one edge 20. In one embodiment, the seal path 26 is a perforated seal that extends linearly between two opposing edges 20. A pulling force exerted on an outer margin edge portion 28 causes the edge portion 28 to separate from the main body 30 of package 10 along the releasable seal path 26. In one embodiment, an adhesive strip 32 including a release liner 34 is disposed on a main body 30 proximal to the edge portion 28. In one embodiment, the release liner 34 is removed from the adhesive strip 32 on one side of the flexible sheet 14 and the open end of the main body 30 is folded over to reseal the package (see, e.g., FIG. 8).

In one embodiment, the inner flexible sheet 16 and the outer flexible sheets 12, 14 are the same material. In one embodiment, at least the flexible sheet 12 and the flexible sheet 16 that form the first compartment 22 are of a moisture-proof material. In one embodiment, a moisture proof liner 36 is provided on the interior surfaces of the first compartment 22. The moisture proof liner 32 can be coated or otherwise suitably applied to the flexible sheets 12, 16. In one embodiment, the flexible sheets 12, 14, 16 are an opaque flexible plastic material. In one embodiment, the flexible sheets 12, 14, 16 all have the same width, length and material thickness.

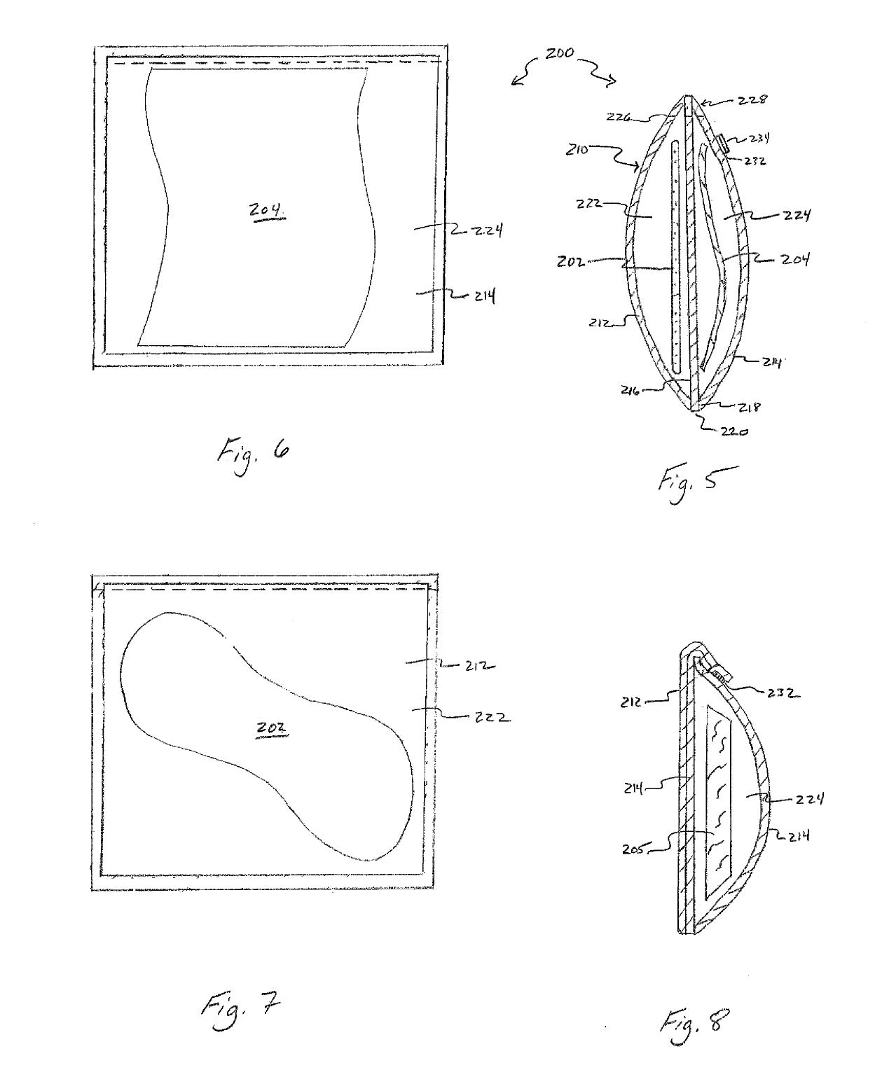

FIGS. 3 and 4 illustrate a medical product package 110 according to one embodiment. The medical product package 110 includes flexible sheets 112, 114, 116 arranged with major surfaces adjacent one another and periphery edges 120 bonded together with a seal 118. The seal 118 can be only at the periphery edges 120 or extend inward from the edges 120 (see, e.g., FIG. 1) to provide a suitably sealed periphery edge 120. A releasable seal path 126 is located parallel to, and inset from, an outer edge 119. An outer margin 128 extends from the releasable seal path 126 and terminates at the outer edge 119 of the flexible sheets 112, 114, 116. In one embodiment, the releasable seal path 126 includes male elements 138 and female elements 139 operatively engageable to effect a latching and unlatching attachment. The fastening elements can be “zip-lock” engageable elements. In one embodiment, the male elements 138 and the female elements 139 on the flexible sheets 112, 114, 116 are aligned with each other to provide closure of a first compartment and a second compartment with a single sealing action. At least the first compartment 122 is moisture proof and prevents ingress or egress of moisture when male element 138 and female element 139 are of the flexible sheets 112 and 116 are engaged.

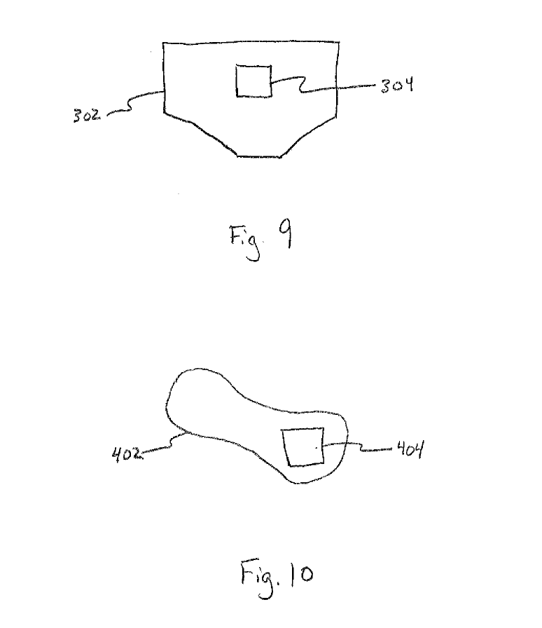

FIG. 5 illustrates a side cross-sectional view of a medical product package assembly 200 according to one embodiment. The medical product package assembly 200 is a disposable kit including a first consumable product 202 in a first compartment 222 of a package 210 and a second consumable product 204 in a second compartment 224 of the package 210. With additional reference to the front view illustrated in FIG. 6, the first consumable product 202 is enclosed in the first compartment of the medical product package assembly 200. The first compartment 222 is moisture proof. The first consumable product 202 is a moist towelette. The towelette can be any type of commercially available pre-moistened disposable towelette suitable to clean urine, feces, or menses from a human body. In one embodiment, the towelette is moistened with an antiseptic solution. The towelette can be a biodegradable organic towelette. The towelette can be flushable. The size of the towelette can vary. In one embodiment, the towelette is scented with a lavender scent, for example.

With reference to the back view illustrated in FIG. 7, the second consumable product 204 is enclosed in the second compartment 224 of the medical product package assembly 200. The second consumable product 204 is a disposable undergarment of any commercially type. The second consumable product 204 can be a disposable sanitary pad or disposable undergarment, including, for example, panty liner, maxi-pad, or disposable underwear.

The size of the assembled packaged kit is suitable to be carried in a purse, for example, or otherwise easily transportable. The size of the assembled package 200 varies as necessary to accommodate the inclusion of a single first consumable product 202 and a single second consumable product 204.

FIGS. 9 and 10 illustrate front views of medical product package assemblies according to embodiments. With reference to FIG. 9, a first consumable product 304, such as a single pre-packaged moist toilette, is removably coupled to a second consumable product 302, such as a disposable undergarment. With reference to FIG. 10, a first consumable product 404, such as a single prepackaged moist toilette, is removably coupled to a second consumable product 402, such as a sanitary napkin. The first consumable product 304, 404, can be removably coupled to the second consumable product 302, 402 by removable adhesive or other suitable manner and can be coupled to any suitable location on the second consumable product 302, 402. The assembly can be placed inside a flexible package, such as package 10 or 110, for example, if desired.

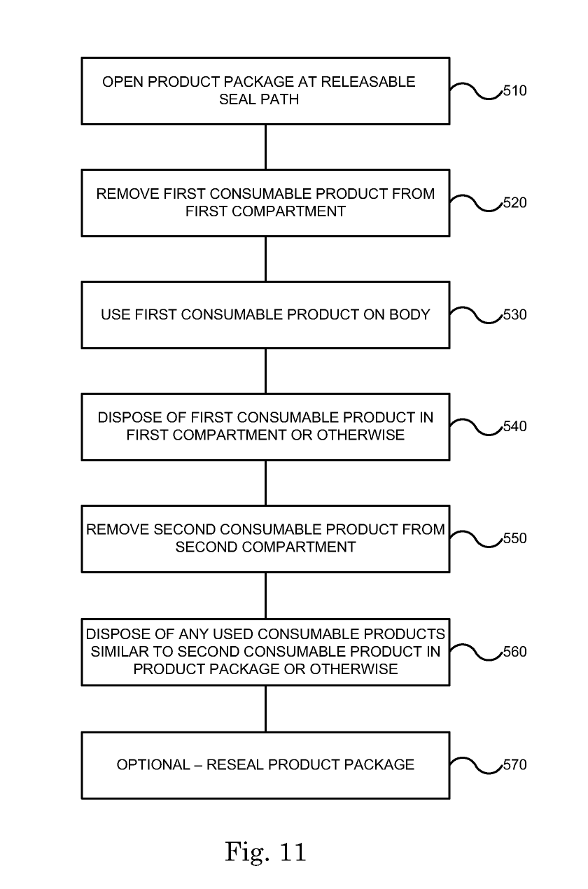

FIG. 11 provides a method of use of a medical product package assembly according to one embodiment. At 510, a product package is opened at a releasable seal path. At 520, a first consumable product is removed from a first compartment of the product package. At 530, the first consumable product is used as needed to clean the desired body area. At 540, the first consumable product is disposed of in the first compartment or otherwise. At 550, a second consumable product is removed from a second compartment of the product package. At 560, a used consumable product similar to the second consumable product is disposed of in the product package or otherwise. Optionally, at 570, the product package is resealed.

In a particular example, a woman with chronic urinary incontinence and a history of urinary tract infections is away from home. The woman wears a pad because she leaks urine when she sneezes or coughs. She carries the medical product package assembly in her purse. She sneezes and leaks urine into her pad that she had put on at the beginning of the day. She does not like this wet feeling and fears that others will smell urine odor from her wet pad. She also fears a urinary tract infection from the stagnant urine. She enters a restroom, opens her purse and takes out the medical product package assembly. She tears opens the package at the releasable seal path and removes the moist towelette from the first compartment. While holding the package in her left hand (she is right handed), she wipes her peritoneum with the towelette in her right hand and throws it in the toilet, if biodegradable and flushable, or disposes of it in the empty first compartment. She disposes of her urine-soaked pad in the first compartment or in a receptacle provided in the restroom stall. She then takes out a dry pad that remains in the second compartment of the package and secures it in her underwear. She disposes of the package in a waste receptacle. She feels clean, confident and ready to continue the many tasks that she has planned for her day.

Although specific embodiments have been illustrated and described herein, it will be appreciated by those of ordinary skill in the art that a variety of alternate and/or equivalent implementations may be substituted for the specific embodiments shown and described without departing from the scope of the present invention. This application is intended to cover any adaptations or variations of the specific embodiments discussed herein. Therefore, it is intended that this invention be limited only by the claims and the equivalents thereof.

What is claimed is:

1. A medical product package assembly comprising:
a package comprising:
an interior flexible sheet disposed between two exterior flexible sheets, the interior and exterior flexible sheets coupled together to form a first compartment and a second compartment wherein the first compartment and the second compartment are adjacently disposed and are formed on opposite sides of the interior flexible sheet; and
a releasable seal path extending across a length of the first and second compartments.

2. The assembly of claim 1, further comprising:
a moist towelette contained within the first compartment.

3. The assembly of claim 1, further comprising:
a consumable product selected from the group of a disposable undergarment and a disposable pad is contained within the second compartment.

4. The assembly of claim 1, wherein the first compartment is moisture-proof

5. The assembly of claim 1, wherein the flexible sheets are opaque.

6. The assembly of claim 1, wherein the releasable seal path is resealable.

7. The assembly of claim 1, wherein the flexible package is biodegradable.

8. The assembly of claim 1, wherein the releasable seal path is a perforated line.

9. The assembly of claim 1, wherein the releasable seal path includes mateable male and female components.

10. A medical package product assembly, comprising:
a flexible package including separate and distinct compartments, wherein the compartments include common perimeter edges and include a common releasably sealed edge, and wherein a first compartment configured to be moisture resistant;
a disposable moist towelette removably contained in the first compartment; and
a disposable undergarment product removably contained in a second compartment.

11. The assembly of claim 10, wherein the first compartment includes a moisture-proof liner.

12. The assembly of claim 10, wherein the flexible package comprises three flexible thin film sheets aligned and sealed together on common edges.

13. The assembly of claim 10, wherein the releasable edge is resealable.

14. The assembly of claim 13, further including an adhesive stripe adjacent to the releasable edge.

15. The assembly of claim 10, wherein the flexible package is biodegradable.

16. A method of using a medical product package assembly, comprising:
opening a product package at a releasable seal path;
removing a first consumable product from a first compartment of the product package;
disposing of the first consumable product; and
removing a second consumable product from a second compartment of the product package.

17. The method of claim 16, further comprising resealing the product package.

18. The method of claim 16, wherein the first consumable product is a moist towelette.

19. The method of claim 16, wherein the second consumable product is a disposable sanitary pad.

20. The method of claim 16, wherein the second consumable product is a disposable undergarment.
